(19) United States
(12) Patent Application Publication
(10) Pub. No.: US 2020/0086065 A1
(43) Pub. Date: Mar. 19, 2020

(54) SPACER FOR A SYRINGE

(71) Applicant: HOFFMANN-LA ROCHE INC., Little Falls, NJ (US)

(72) Inventors: Christopher Harold MUENZER, Basel (CH); Neil B. CAMMISH, Basel (CH); Jörg SIELEMANN, Basel (CH)

(21) Appl. No.: 16/470,498

(22) PCT Filed: Dec. 15, 2017
(86) PCT No.: PCT/EP2017/083124

§ 371 Date: Jun. 17, 2019

(30) Foreign Application Priority Data
Dec. 16, 2016 (EP) 16204 604.9

(51) Int. Cl. A61M 5/46; A61M 5/178

(52) U.S. Cl. CPC A61M 5/46; A61M 5/178

(57) ABSTRACT

A spacer for a syringe for manual injection includes a mount section, a flange section and a stopper section. The mount section is adapted to be coupled to a distal end of a syringe body of the syringe. The flange section includes a contact face adapted to contact a skin surface of a patient. The stopper section has a bore extending between the mount section and the flange section wherein the stopper section is adapted to define an injection depth of a needle when being attached to the distal end of the syringe body of the syringe and extending through the bore of the stopper section.

### TECHNICAL FIELD

The present invention relates to a spacer for a syringe for manual injection as well as to a kit comprising a spacer and a syringe.

### BACKGROUND ART

In medical or therapeutic applications pharmaceutical products or drugs are administered to patients many different ways. Some drugs require injection by means of wherein typically it is important where the drug is injected. In particular, some drugs have to be injected into a specific layer of the skin of a patient for being properly administered. Depending on the kind of drug intradermal or subcutaneous injections are desired.

For facilitating that drugs are properly injected there are specific devices known wherein it can still be rather challenging to locate and reach the spot such as the right layer of the skin. For patients injecting drugs by themselves, so-called auto-injectors such as EpiPens are widespread. Such auto-injectors are syringes, which are designed to automatically or semi-automatically deliver a dose of a drug, e.g. via or into the skin. The angle of the needle in regard to the skin surface as well as the injection depth is controlled by the device.

For clinical studies, auto-injectors are often not appropriate since, e.g., it can be disproportionally expensive to produce comparably small quantities of auto-injectors as required for such studies. Therefore, conventional manual syringes for manual injections are typically used instead of auto-injectors. However, when using conventional manual syringes the results of the clinical studies can be impaired. For example, the depth of insertion, the angle of the needle and/or the skin contact effects can differ in manual injection.

It is therefore an object of the present invention to provide a comparably simple and efficient device to allow mimicking characteristics of an auto-injector when using a conventional syringe for manual injection.

### DISCLOSURE OF THE INVENTION

According to the invention this need is settled by a spacer as it is defined by the features of independent claim 1 and by a kit as it is defined by the features of independent claim 11. Preferred embodiments are subject of the dependent claims.

In particular, the invention deals with a spacer for a syringe for manual injection, comprising a mount section adapted to be coupled to a distal end of a syringe body of the syringe, a flange section comprising a contact face adapted to contact a skin surface of a patient, and a stopper section having a bore extending between the mount section and the flange section. The stopper section is adapted to define an injection depth of a needle when the spacer is attached to the distal end of the syringe body of the syringe and the needle extends through the bore of the stopper section.

The term “distal end” in connection with the syringe relates to a side of the syringe body which is directed towards a patient when injecting. Typically, the distal end of the syringe body is equipped with a needle or a connector for mounting a needle. For example, the connector can be a male or female part of a so-called Luer Lock system. Thereby, Luer Lock system is preferably not affected by the mount section of the spacer or any other part thereof.

The syringe body can be cylindrical and shaped to house a fluid to be injected. By means of the mount section, the spacer is couplable or pluggable to the distal end of the syringe body. The mount section can, thus, be easily or conveniently attached to the syringe such as, e.g., a disposable or non-disposable plastic or glass syringe. The mount section of the spacer can for example surround the needle and/or the Luer Lock system.

The spacer can particularly be a one piece construction, e.g., made of a plastic material. The design of the spacer can be comparably simple and cost efficient. Therefore, the spacer can also be used, e.g., in developing countries.

For defining the injection depth of the needle of the syringe, the stopper section or its bore can have a fixed or predefined length such that it cannot be adjusted. The stopper section can hide a part of the needle. The injection depth can correspond to a length of the stopper section as the stopper section is adapted to provide a defined space between the distal end of the syringe body and the part of the needle capable for penetration. For example, the part of the needle capable for penetration when the spacer is mounted to the syringe preferably has a length between about 5 mm and about 7 mm. The spacer, thus, can be arranged for subcutaneous injection. However, if a longer stopper section is used, the part of the needle capable for penetration can be reduced to less than 2 mm. Like this, intradermal injection is possible.

The flange section can, e.g., be ring-shaped or angled. It can surround the needle when the spacer is mounted to the syringe, whereby the needle can in particular be arranged centrally. As the spacer does not contact the needle contaminations can be prevented. The contact face can form a flat or inclined level. Thereby, an injection angle can depend on the inclination of the contact area. As the injection angle is predefined by the flange section, misuse of the syringe can be prevented such that even un-trained patients can inject in the required angle. In particular, the contact face can be designed in a similar way as the contact area of an auto-injector to mimic.

In a particularly efficient embodiment, the stopper section is arranged coaxial to a longitudinal axis of the syringe body when the spacer is mounted to the syringe. Also, the flange section can preferably be arranged coaxial to the longitudinal axis of the syringe body when the spacer is mounted to the syringe.

When being applied the spacer allows for mimicking conditions or circumstances similar to those of auto-injectors. In particular, the injection depth can be predefined by dimensioning the bore and the stopper section accordingly. Additionally, the contact face allows for ensuring that the needle is correctly oriented with regard to the surface of the skin. Also, it allows for distributing the pressure applied when forwarding a rod of the syringe over a comparably large area of the skin such that deformation of the skin can be reduced. Like this, the spacer allows for ensuring that a conventional syringe is properly applied, i.e. at a correct angle and to a correct piercing depth.

Thus, similar to known auto-injectors, the spacer allows for conveniently and accurately delivering a substance to a predefinable location in the body of the patient by means of a conventional syringe. Therefore, with the help of the spacer, it is possible to mimic an auto-injector, e.g., its injection depth, its needle angle, and/or the skin contact. Therefore, a conventional syringe with the spacer can be used instead of an auto-injector for clinical studies as well as for normal use. This allows for reducing the effort of clinical studies and/or for ensuring an appropriate injection.

Preferably, a thickness of the flange section broadens from the stopper section to the contact face. Like this, a comparably thick or large contact face can be provided in an efficient manner. This allows for enhancing the area in which the spacer contacts the skin when the syringe is applied to the patient. Thus, a better pressure distribution can be achieved and deformations of the skin can be reduced. Furthermore, the syringe can be positioned on the skin surface of a patient in a stable way which may enable an easy handling during injection. Still further, the broadened contact face can mimic characteristics of an auto-injector.

Thereby, the thickness of the flange section at the contact face is advantageously larger than a wall thickness of the syringe body. It can be at least about 2 millimeters (mm), at least about 3 mm, at least about 4 mm or at least about 5 mm.

Preferably, the flange section is adapted to ensure a perpendicular orientation of the syringe to a skin surface of the patient. In other words, it can be adapted to only permit penetrations perpendicular to the skin surface of the patient. Like this, a correct orientation of the syringe desired in many applications can be ensured.

Preferably, the bore of the stopper section has an essentially straight central axis, the contact face of the flange section is essentially plain and the central axis of the bore of the stopper section is arranged essentially perpendicular to the contact face of the flange section. Thereby, the plain contact face can form a flat level which can be parallel to the skin surface and/or perpendicular to the needle of the syringe. Like this, it can efficiently be achieved that the spacer and thus the syringe stand upright in regard to the skin surface of the patient. The injection angle can automatically be adjusted at 90°. This enables even untrained persons to correctly inject drugs in a predefined injection angle of about 90°. Also, such fixed injection angle can mimic characteristics of an auto-injector.

Preferably, the mount section, the stopper section and/or the flange section are configured quasi as a sleeve. In particular, the complete spacer can be configured as a sleeve. Thereby, the sleeve preferably is at least partial cylindrical which allows the spacer to be efficiently manufactured and to be particularly suitable for cylindrical syringe bodies.

Alternatively, only one or more parts of the spacer, i.e. the mount section, the stopper section or the flange section, can be configured as a sleeve.

The sleeve-shaped mount section may allow the spacer for being conveniently attached to the distal end of a cylindrical syringe body. It can cover the whole circumference of the syringe and thus provide a safe fixing.

The sleeve-shaped stopper section can efficiently define a constant distance between the distal end of the syringe body and the skin surface of a patient.

The sleeve-shaped flange can touch the skin surface of the patient uniformly. During injection, the pressure to the skin can thus be equally allocated over the whole contact face.

Preferably, an outer diameter of the flange section at the contact face exceeds an outer diameter of the stopper section. Like this, the flange section, thus, enlarges the contact face abutting the skin surface. Therefore, the syringe can be placed at the skin in a stable way. Also, the smaller outer diameter of the stopper section can improve the handling, as it can be seen from outside which part of the spacer has to be coupled to the distal end of the syringe body and which part has to be brought in contact with the skin surface.

An outer diameter of the mount section preferably is equal to an outer diameter of the stopper section. From outside, a transition between the mount and the stopper can be smooth and these parts of the spacer may appear as one unit.

In particular, a transition area between the stopper section and the flange section preferably is conical. Starting from the stopper section or even the mount section, the spacer can enlarge or enhance towards to the flange section or its contact face. Such transition can be particularly smooth. Such conical form can contribute to achieving high hygienic standards as the outer surface has no undercuts. The spacer may, thus, be reused after cleaning.

In an embodiment of the spacer, an inner diameter of the mount section is smaller than an inner diameter of the stopper section and/or the flange section. As the syringe bodies of many syringes comprise a recessed area at the distal end, the syringe body can be coupled to the mount section via such recessed area. The inner diameter of the mount section is smaller in order to positively lock with the recessed area. The inner diameter of the stopper section and/or the flange section can be larger in order to provide enough space for the needle, a protective cover of the needle or a needle shield and/or the Luer Lock system.

Preferably, the mount section comprises an inner flange to form a seat for receiving the distal end of the syringe body of the syringe. Thereby, the inner flange can have the smallest inner diameter of the spacer. It can be ring-shaped to provide a uniform strain as the seat. Such a seat, which may provide a smaller inner diameter of the mount section as mentioned above, allows for efficiently receiving the syringe body at a predefined position and orientation. Thus, the syringe can conveniently and precisely be coupled to the spacer.

Preferably, in a plane defined by the contact face of the flange section, an area of the bore is about 1 times to about 5 times or about 1.3 times to about 4 times or about 1.5 times to about 3 times larger than an area of the contact face. Such a relation between the cross sectional bore area and contact face area can allow for efficiently providing an advantageously dimensioned contact face.

Another aspect of the invention relates to a kit comprising a syringe and a spacer as described above. Such a kit allows for efficiently providing the effects and benefits mentioned in connection with spacer and its preferred embodiments.

In the kit, the spacer can be pre-coupled to the distal end of the syringe body of the syringe already. Alternatively, the syringe and the spacer can be separate. In this case, the patient or the person executing the injection couples the spacer before using the syringe.

The kit can comprise several different spacers. In particular, it can comprise spacers with stoppers having different lengths. In use an appropriate spacer can be chosen in consideration of the desired injection depth of the needle of the syringe.

Preferably, the stopper section of the spacer of the kit is adapted to provide a defined space between a distal end of a syringe body of the syringe of the kit and a portion of a needle attached to the distal end of the syringe body of the syringe capable for penetration. Spacers having different stopper sections can be used in order to adjust the desired injection depth of the needle. Thereby, the part of the needle capable for penetration preferably has a length between about 5 mm and about 7 mm. The spacer can thereby enable, for example, a subcutaneous injection. However, if a longer stopper is used, the part of the needle capable for penetration can be reduced to less than 2 mm. Thereby, e.g., an intradermal injection is possible.

In an embodiment of the kit, the outer diameter of the flange section of the spacer is equal to or larger than the outer diameter of a fluid chamber of the syringe body. Like this, the flange section can provide a comparably large contact area which may stabilize the syringe during injection.

Preferably, the kit further comprises an extended finger flange adapted to be coupled to a proximal end of the syringe. Conventional syringes often comprise a comparably small finger flange which is not easy to handle particularly for handicapped people. And some syringes don't comprise finger flanges at all. The extended finger flange can, e.g., be clipped, screwed or locked to the syringe body and/or the conventional finger flange of the syringe. The extended finger flange thus enlarges the conventional finger flange or establishes a finger flange. This can make the handling of the syringe easier especially for handicapped people.

### BRIEF DESCRIPTION OF THE DRAWINGS

The spacer according to the invention and the kit according to the invention are described in more detail herein below by way of an exemplary embodiment and with reference to the attached drawings, in which:

FIG. 1 shows a perspective view of an embodiment of a kit according to the invention comprising a syringe and an embodiment of a spacer according to the invention;

FIG. 2 shows an exploded perspective view of the syringe and the spacer of FIG. 1;

FIG. 3 shows a cross sectional view of the syringe and the spacer of FIG. 1; and

FIG. 4 shows an enlarged cross sectional view of a distal end of the syringe of FIG. 3.

### DESCRIPTION OF EMBODIMENTS

In the following description certain terms are used for reasons of convenience and are not intended to limit the invention. The terms “right”, “left”, “up”, “down”, “under” and “above” refer to directions in the figures. The terminology comprises the explicitly mentioned terms as well as their derivations and terms with a similar meaning. Also, spatially relative terms, such as “beneath”, “below”, “lower”, “above”, “upper”, “proximal”, “distal”, and the like, may be used to describe one element's or feature's relationship to another element or feature as illustrated in the figures. These spatially relative terms are intended to encompass different positions and orientations of the devices in use or operation in addition to the position and orientation shown in the figures. For example, if a device in the figures is turned over, elements described as “below” or “beneath” other elements or features would then be “above” or “over” the other elements or features. Thus, the exemplary term “below” can encompass both positions and orientations of above and below. The devices may be otherwise oriented (rotated 90 degrees or at other orientations), and the spatially relative descriptors used herein interpreted accordingly. Likewise, descriptions of movement along and around various axes include various special device positions and orientations.

To avoid repetition in the figures and the descriptions of the various aspects and illustrative embodiments, it should be understood that many features are common to many aspects and embodiments. Omission of an aspect from a description or figure does not imply that the aspect is missing from embodiments that incorporate that aspect. Instead, the aspect may have been omitted for clarity and to avoid prolix description. In this context, the following applies to the rest of this description: If, in order to clarify the drawings, a figure contains reference signs which are not explained in the directly associated part of the description, then it is referred to previous or following description sections. Further, for reason of lucidity, if in a drawing not all features of a part are provided with reference signs it is referred to other drawings showing the same part. Like numbers in two or more figures represent the same or similar elements.

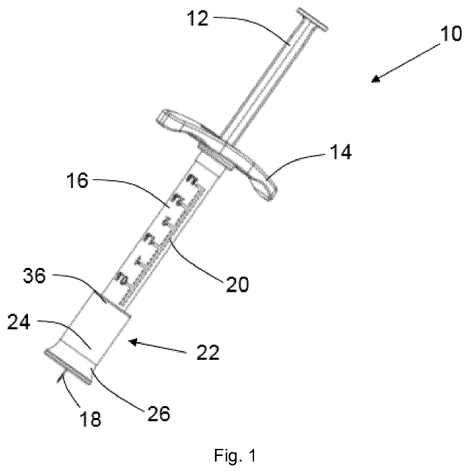

In FIG. 1 an embodiment of a kit according to the invention is shown. The kit comprises a syringe 10, an extended finger flange 14 and an embodiment of a spacer 22 according to the invention. The syringe 10 has a cylindrical syringe body 16, a plunger rod 12 extending into the syringe body 16 through an open proximal end thereof and a needle 18 mounted to a distal end of the syringe body 16. In the interior of the syringe 10 a fluid chamber is formed where a drug is stored before injection. The syringe body 16 has a scale 20 showing the amount of fluid inside the syringe body 16. It is made of a plastic material or of glass and advantageously is transparent. The extended finger flange 14 is mounted to the proximal end of the syringe body 16 where the plunger rod 12 enters the interior of the syringe body 16.

The spacer 22 is a single piece construction comprising a stopper section 24, a mount section 36 and a flange section 26. It has a generally cylinder-like shape and is made of a plastic material. The spacer 22 is coupled to the distal end of the syringe body 16. The stopper section 24 has a longitudinal or axial through hole or bore through which the needle 18 of the syringe 10 is arranged. It extends from the flange section 26 to the mount section 36 and defines an injection depth of the needle 18.

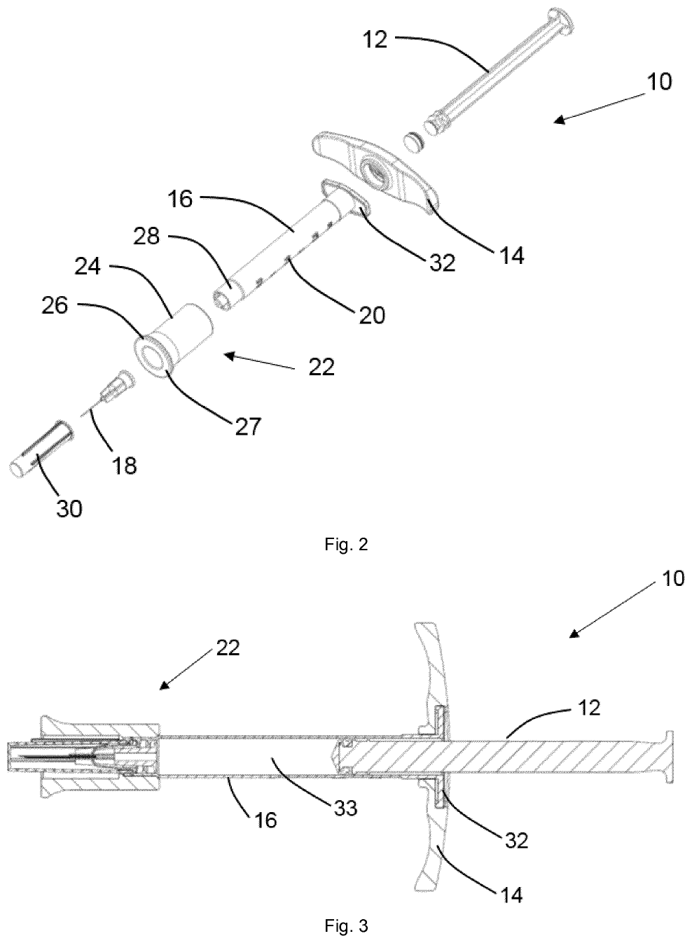

As can be seen in FIG. 2, the flange section 26 of the spacer 22 comprises a plain, ring-shaped contact face 27 or contact area. The flange section 26 is conically shaped in an axial direction such that the contact face 27 is comparably wide in order to provide a stable positioning for the syringe 10 on the skin surface of a patient and in order to allow for an enhanced pressure distribution to the skin when injecting.

The distal end of the syringe body 16 has a recessed area 28 to which the spacer 22 is couplable. Prior to the syringe 10 being applied the needle 18 is covered by a protective cover 30 or needle shield. For example, during transportation, storage, and/or during the mounting of the spacer 22 the needle 18 is protected. The risk of injury or of contamination of the needle 18 is, thus, minimized. Furthermore, the needle 18 is kept under sterile conditions.

The syringe 10 comprises a small, conventional finger flange 32. The extended finger flange 14 of the kit is mounted, e.g. screwed or clipped, at the conventional finger flange 32. An easier or improved handling is thus provided. The extended finger flange 14 can also be seen in the cross sectional view of FIG. 3. The broadness of the conventional finger flange 32 is at least doubled by the extended finger flange 14.

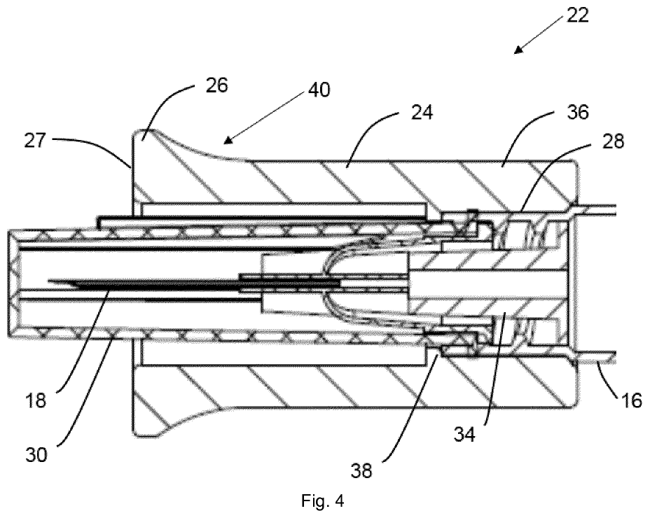

FIG. 4 shows an enlarged view of the distal end of the syringe body 16 of the syringe 10. The needle 18 is locked to the syringe body 16 by a Luer Lock System 34. The Luer Lock System 34 is preferably arranged inside the recessed area 28 of the syringe body 16. The Luer Lock System 34, the needle 18, and the protective cover 30 are surrounded by the spacer 22.

The mount section 36 of the spacer 22 is configured as a sleeve and couples the spacer 22 to the recessed area 28 of the syringe body 16. The mount section 36 comprises an inner flange 38 as a limit stop for the distal end of the syringe body 16. The stopper section 24 defines the injection depth of the needle 18. The longer the stopper section 24 is configured, the shorter the injection depth of the needle 18 is.

An inner diameter of the stopper section 24 is bigger than an inner diameter of the mount section 36, whereas outer diameters of these parts are equal. An outer diameter of the flange section 26 at the distal end of the spacer 22 is bigger than the outer diameter of the stopper section. A transition area 40 between the stopper section 24 and the flange 26 is formed conical. The flange section 26 provides the comparably large contact face 27 similarly dimensioned as a contact area of an auto-injector.

In use, the spacer 22 can easily be coupled to the distal end of the syringe body 16. The protective cover 30 of the needle is removed. The contact face 27 provides a stable, upright position of the syringe 10 at about 90° to the skin, whereas the stopper 24 section defines the desired injection depth. With the help of the spacer 22, several characteristics of an auto-injector can thus be mimicked in a simple way.

This description and the accompanying drawings that illustrate aspects and embodiments of the present invention should not be taken as limiting-the claims defining the protected invention. In other words, while the invention has been illustrated and described in detail in the drawings and foregoing description, such illustration and description are to be considered illustrative or exemplary and not restrictive. Various mechanical, compositional, structural, electrical, and operational changes may be made without departing from the spirit and scope of this description and the claims. In some instances, well-known circuits, structures and techniques have not been shown in detail in order not to obscure the invention. Thus, it will be understood that changes and modifications may be made by those of ordinary skill within the scope and spirit of the following claims. In particular, the present invention covers further embodiments with any combination of features from different embodiments described above and below.

The disclosure also covers all further features shown in the Figs. individually although they may not have been described in the afore or following description. Also, single alternatives of the embodiments described in the figures and the description and single alternatives of features thereof can be disclaimed from the subject matter of the invention or from disclosed subject matter. The disclosure comprises subject matter consisting of the features defined in the claims or the exemplary embodiments as well as subject matter comprising said features.

Furthermore, in the claims the word “comprising” does not exclude other elements or steps, and the indefinite article “a” or “an” does not exclude a plurality. A single unit or step may fulfil the functions of several features recited in the claims. The mere fact that certain measures are recited in mutually different dependent claims does not indicate that a combination of these measures cannot be used to advantage. The terms “essentially”, “about”, “approximately” and the like in connection with an attribute or a value particularly also define exactly the attribute or exactly the value, respectively. The term “about” in the context of a given numerate value or range refers to a value or range that is, e.g., within 20%, within 10%, within 5%, or within 2% of the given value or range. Components described as coupled or connected may be electrically or mechanically directly coupled, or they may be indirectly coupled via one or more intermediate components. Any reference signs in the claims should not be construed as limiting the scope.

1. A spacer for a syringe for manual injection, comprising:
a mount section adapted to be coupled to a distal end of a syringe body of the syringe;
a flange section comprising a contact face adapted to contact a skin surface of a patient; and
a stopper section having a bore extending between the mount section and the flange section, wherein the stopper section is adapted to define an injection depth of a needle when the spacer is attached to the distal end of the syringe body of the syringe and the needle extends through the bore of the stopper section.

2. The spacer according to claim 1, wherein a thickness of the flange section broadens from the stopper section to the contact face.

3. The spacer according to claim 1, wherein the flange section is adapted to ensure a perpendicular orientation of the syringe to a skin surface of the patient during injection.

4. The spacer according to claim 1, wherein the bore of the stopper section has an essentially straight central axis, the contact face of the flange section is essentially plain and the central axis of the bore of the stopper section is arranged essentially perpendicular to the contact face of the flange.

5. The spacer according to claim 1, wherein the mount section, the stopper section and/or the flange section is configured quasi as a sleeve.

6. The spacer according to claim 5, wherein the sleeve is at least partial cylindrical.

7. The spacer according to claim 1, wherein an outer diameter of the flange section at the contact face exceeds an outer diameter of the stopper section.

8. The spacer according to claim 1, wherein a transition area between the stopper section and the flange section is conical.

9. The spacer according to claim 1, wherein the mount section comprises an inner flange to form a seat for receiving the distal end of the syringe body of the syringe.

10. The spacer according to claim 1, wherein in a plane defined by the contact face of the flange section an area of the bore is about 1 times to about 5 times, or about 1.3 times to about 4 times, or about 1.5 times to about 3 times, larger than an area of the contact face.

11. A kit comprising
a syringe, and
a spacer according to claim 1.

12. The kit according to claim 11, wherein the stopper section of the spacer is adapted to provide a defined space between a distal end of a syringe body of the syringe and a portion of a needle attached to the distal end of the syringe body of the syringe capable for penetration.

13. The kit according to claim 12, wherein the part of the needle capable for penetration has a length between about 5 mm and about 7 mm.

14. The kit according to claim 11, further comprising an extended finger flange adapted to be coupled to a proximal end of the syringe.
